(19) United States
(12) Patent Application Publication
(10) Pub. No.: US 2025/0382209 A1
(43) Pub. Date: Dec. 18, 2025

(54) METHOD FOR ENHANCING SELF-ENRICHMENT OF ANAMMOX BACTERIA BY SYMBIOTIC METABOLISM OF NITRATE-DEPENDENT DENITRIFYING BACTERIA

(71) Applicant: Beijing University of Technology, Beijing (CN)

(72) Inventors: Rui Du, Beijing (CN); Fangyu Chang, Beijing (CN); Shenbin Cao, Beijing (CN); Yongzhen Peng, Beijing (CN)

(21) Appl. No.: 19/031,489

(22) Filed: Jan. 18, 2025

(30) Foreign Application Priority Data
Jun. 13, 2024 (CN) 202410763445.9

(51) Int. Cl. C02F 3/28; C02F 3/00; C02F 3/34; C02F 101/16; C12Q 1/686; C12Q 1/689

(52) U.S. Cl. CPC C02F 3/282; C02F 3/006; C02F 3/2866; C02F 3/34; C12Q 1/686; C12Q 1/689; C02F 2101/16; C02F 2209/10; C02F 2209/14; C02F 2209/15; C02F 2209/20; C02F 2209/225; C02F 2209/38; C02F 2305/06

(57) ABSTRACT

Provided is a method for enhancing self-enrichment of anammox bacteria by nitrate-dependent denitrifying bacteria symbiotic metabolism, belonging to the field of biological wastewater disposal. Excess sludge from a municipal wastewater treatment plant is inoculated into an enhanced anammox bacteria self-enrichment system (a closed SBR (Sequencing Batch Reactor)). The method includes the following steps: taking sodium acetate as a carbon source, enriching nitrate-dependent denitrifying bacteria at first, and transforming nitrate into nitrite to provide a sufficient substrate for the growth of anammox bacteria; in turn, the enriched anammox bacteria provide nitrate for the nitrate-dependent denitrifying bacteria to grow.

### CROSS-REFERENCE TO RELATED APPLICATION

This patent application claims the benefit and priority of Chinese Patent Application No. 202410763445.9 filed with the China National Intellectual Property Administration on Jun. 13, 2024, the disclosure of which is incorporated by reference herein in its entirety as part of the present application.

### TECHNICAL FIELD

The present disclosure provides a method for enhancing self-enrichment of anammox bacteria by nitrate-dependent denitrifying bacteria symbiotic metabolism, belonging to the field of biological wastewater treatment. The method is suitable for the self-enrichment of anammox bacteria in an activated sludge system to achieve advanced nitrogen removal of municipal wastewater, featuring the advantages of energy saving and consumption reduction.

### BACKGROUND

At present, with the continuous growth of urban populations and the accelerated urbanization, the amount of municipal wastewater is also increasing. In addition, there is a global reduction in freshwater resources due to urbanization, making the wastewater recycle and reuse necessary and urgent. Nitrogen is a major pollutant in municipal wastewater, and nitrogen removal is one of the most important issues in the wastewater treatment plants. Compared with the physical-chemical process, biological nitrogen removal is a cost-effective and energy-saving method that is less likely to produce secondary pollution. Given the global goals on carbon peaking and carbon neutrality, biological nitrogen removal is the most acceptable choice for municipal wastewater treatment.

For biological nitrogen removal, anaerobic ammonium oxidation (anammox) technology is regarded as the most potential nitrogen removal process because of its high efficiency and low energy consumption. Under anaerobic conditions, anammox bacteria can directly oxidize ammonium into nitrogen gas with nitrite as an electron acceptor, which can remove nitrite and ammonium at the same time. The process has the advantages of no need for aeration and external organic carbon source, less excess sludge production, and no secondary pollution. It thus holds a great potential for treating wastewater containing high ammonium, especially wastewater with low carbon-to-nitrogen (C/N) ratio. However, the mainstream application of anammox technology has not yet been achieved. This is primarily due to two challenges. Firstly, there is a lack of stable nitrite generation. Secondly, the slow growth-rate of anammox bacteria results in the challenge for their enrichment and retention. At present, small-scale experimental anammox technology applications mainly rely on inoculating pre-cultivated anammox sludge. However, in practical large-scale applications, the long sludge cultivating period and the large quantity of sludge required make inoculation challenging.

In order to solve the above problems, a partial denitrification coupled anammox process is proposed by self-enrichment of anammox bacteria in activated sludge systems. This emerges as a new approach providing nitrite for anammox bacteria, and can get rid of the dependence on inoculation of anammox seeding sludge. In this coupling system, nitrate-dependent denitrifying bacteria and anammox bacteria coexist and metabolize symbiotically. This prevents the denitrifying bacteria from excessively competing for substrates and living space with anammox bacteria. Meanwhile, it ensures adequate supply of nitrite to facilitate the growth of the anammox bacteria, thereby promoting the self-enrichment of anammox bacteria. Denitrifying bacteria can convert organic matter into substrates that can be utilized by anammox bacteria, such as acetic acid and propionic acid, which promotes the growth and reproduction of anammox bacteria. Additionally, denitrifying bacteria can degrade or synthesize extracellular substances, and secrete secondary metabolites to interact with the anammox bacteria, promoting the activity and growth of anammox bacteria. Furthermore, due to the much slower growth rate of anammox bacteria compared to denitrifying bacteria, anammox bacteria are easily washed out of the system in the effluent. By integrating a granule recycling system behind the coupling system in series, anammox bacteria can be effectively retained, thus enhancing the self-enrichment of anammox bacteria, and achieving advanced nitrogen removal performance.

### SUMMARY

The present disclosure proposes a method for enhancing self-enrichment of anammox bacteria by nitrate-dependent denitrifying bacteria symbiotic metabolism. Specifically, the method includes inoculating excess sludge from municipal wastewater treatment plants into an enhanced anammox bacteria self-enrichment system (a closed SBR (Sequencing Batch Reactor)); with sodium acetate as a carbon source, enriching nitrate-dependent denitrifying bacteria at first, and converting nitrate into nitrite to provide a sufficient substrate for anammox bacteria. In turn, the enriched anammox bacteria produces nitrate for nitrate-dependent denitrifying bacteria. By adjusting key factors, including C/N ratio and anoxic reaction time, as well as further integrating a gas-liquid separation system and a hydraulic cyclone separation system in series for linkage, recycled granular sludge is returned to the enhanced self-enrichment system to retain functional bacteria. This ensures that the symbiotic metabolism of nitrate-dependent denitrifying bacteria stimulates the self-enrichment of anammox bacteria, thereby achieving more effective nitrogen removal.

Provided is a device for enhancing self-enrichment of anammox bacteria by nitrate-dependent denitrifying bacteria symbiotic metabolism, including:
an influent tank (1), an enhanced anammox bacteria self-enrichment system (2), an effluent tank (3), an online real-time control system (4), a gas-liquid separation system (5), and a hydraulic cyclone separation system (6);
the enhanced anammox bacteria self-enrichment system (2) is provided with a first wastewater inlet (2.1), a first peristaltic pump (2.2), a first time switch (2.3), a carbon source tank (2.4), a carbon source inlet (2.5), a second peristaltic pump (2.6), a second time switch (2.7), a stirrer (2.8), a third time switch (2.9), an effluent outlet (2.10), an effluent control valve (2.11), a sampling port (2.12), a first probe socket (2.13), a second probe socket (2.14), an overflow pipe (2.15), and a recycled granules inlet (2.16). The online real-time control system is provided with a display screen (4.1), a nitrate probe monitor (4.2), an ammonium probe monitor (4.3), and a dissolved oxygen probe monitor (4.4). The gas-liquid separation system (5) is provided with a mixture inlet (5.1), a gas outlet (5.2), a gas collector (5.3), a gas flow detector (5.4), and a discharge outlet (5.5). The hydraulic cyclone separation system (6) is provided with a U-shaped pipe (6.1), a first valve (6.2), a cyclone inlet (6.3), an outer cyclone (6.4), an inner cyclone (6.5), a granule discharge port (6.6), a second valve (6.7), and an effluent outfall (6.8);
the influent tank (1) is connected to the first wastewater inlet (2.1) of the enhanced anammox bacteria self-enrichment system (2) through the first peristaltic pump (2.2), which is controlled by the first time switch (2.3); the carbon source tank (2.4) is connected to the carbon source inlet (2.5) of the enhanced anammox bacteria self-enrichment system (2) through the second peristaltic pump (2.6), which is controlled by the second time switch (2.7); the stirrer (2.8) is controlled by the online real-time control system (4) and is connected by a wire; the nitrate probe monitor (4.2) is connected to the enhanced anammox bacteria self-enrichment system (2) through the first probe socket (2.13); the ammonium probe monitor (4.3) is connected to the enhanced anammox bacteria self-enrichment system (2) through the second probe socket (2.14); the dissolved oxygen probe monitor (4.4) is connected to the enhanced anammox bacteria self-enrichment system (2) through the sampling port (2.12), which is used to monitor the dissolved oxygen concentration that is controlled below 0.1 mg/L for well anoxic conditions; the enhanced anammox bacteria self-enrichment system (2) is connected to the gas-liquid separation system (5) through the effluent outlet (2.10) and the mixture inlet (5.1), and the process is generated through the effluent control valve (2.11), which is controlled by the third time switch (2.9); the gas-liquid separation system (5) is connected to the gas collector (5.3) through the gas outlet (5.2); the hydraulic cyclone separation system (6) is connected to the gas-liquid separation system (5) through the U-shaped pipe (6.1), which is controlled by the first valve (6.2); the hydraulic cyclone separation system (6) is connected to the enhanced anammox bacteria self-enrichment system (2) through the recycled granules inlet (2.16), which is controlled by the second valve (6.7); the hydraulic cyclone separation system (6) is connected to the effluent tank (3) through the effluent outfall (6.8).

Further provided is a method for enhancing self-enrichment of anammox bacteria by nitrate-dependent denitrifying bacteria symbiotic metabolism, including the following steps:
(1) enrichment of nitrate-dependent denitrifying bacteria: inoculating excess sludge from a municipal wastewater treatment plant into an enhanced anammox bacteria self-enrichment system, where the suspended solids (SS) have a concentration of 3000-6000 mg/L, and volatile suspended solids (VSS) have a concentration of 2000-4000 mg/L; adjusting a mass concentration of nitrate in the influent tank to 50-100 mg/L, and adding sodium acetate as an external carbon source, and performing running cycles in the enhanced anammox bacteria self-enrichment system, where each cycle including influent, carbon source addition, anoxic stirring, settling, discharging and idling; after the influent and the carbon source addition, a C/N ratio in the enhanced anammox bacteria self-enrichment system is kept at 2.0-3.5; a drainage ratio is 40-70%; the anoxic stirring is performed at 80-140 r/min for 2-4 h; the nitrate concentration is monitored through a nitrate probe monitor during running, and data is uploaded to the online real-time control system; when the anoxic stirring duration reaches 4 h and the nitrate concentration is over 10 mg/L, the C/N ratio is increased in the next cycle; when the anoxic stirring duration reaches 2 h and the nitrate concentration is below 1 mg/L, the C/N ratio is reduced in the next cycle; a condition where a nitrate-to-nitrite transformation ratio exceeds 70% and the effluent nitrate is below 3 mg/L is maintained stably for over 30 days; a community structure of activated sludge is detected and analyzed by 16SrRNA high-throughput sequencing technology; and when a relative abundance of one genus of denitrifying bacteria reaches over 30%, it is determined that the enrichment of nitrate-dependent denitrifying bacteria is successfully achieved;
(2) in situ self-enrichment of anammox bacteria: replacing influent wastewater of the enhanced anammox bacteria self-enrichment system with actual municipal wastewater and nitrate-containing wastewater, where a nitrate concentration in the nitrate-containing wastewater is 50-100 mg/L, and the ammonium concentration in municipal wastewater is 30-80 mg/L; adjusting the C/N ratio to 2.0-2.8; extending the anoxic stirring duration in the enhanced anammox bacteria self-enrichment system to 6-10 h, where when a removal rate of nitrite and ammonium reaches over 80% and 60%, respectively, the effluent nitrate is below 5 mg/L, and the abundance of anammox bacteria is over 104 copies/dry sludge as detected by polymerase chain reaction (PCR), the self-enrichment of anammox bacteria is regarded as achieved; recycling granular sludge discharged from the effluent through the gas-liquid separation system and the hydraulic cyclone separation system connected to the enhanced anammox bacteria self-enrichment system, and returning the granular sludge discharged from the effluent to the self-enrichment enhancement system to retain anammox bacteria; the effluent enters the gas-liquid separation system at first; according to different centrifugal forces on different phases, the gas is discharged upward into a collector equipped with a gas flowmeter for gas generation rate detection; the first valve is opened to have liquid phase enter the hydraulic cyclone separation system through a U-shaped pipe; due to different inertia, flocs enter an inner cyclone separator with a light water phase and is discharged from an upper part into the effluent tank; the granular sludge enters an outer cyclone separator, and flows out from the bottom; and the second valve is opened to recycle and return the granular sludge into the enhanced anammox bacteria self-enrichment system; and
(3) enhanced self-enrichment of anammox bacteria by nitrate-dependent denitrifying bacteria symbiotic metabolism: transiting the anammox bacteria to a self-enrichment enhancement stage, adjusting the concentration of the nitrate-containing influent to 80-200 mg/L, the ammonium concentration to 60-120 mg/L, and the C/N ratio to 2.0-2.5, and performing the anoxic stirring for 3-8 h, where when the concentration of ammonium is 5 mg/L or more and the concentration of nitrate is 1 mg/L or less as monitored by an ammonium probe monitor and a nitrate probe monitor, the nitrate concentration in the influent is decreased in the next cycle until a concentration of total inorganic nitrogen (TIN) is below 5 mg/L; when the concentration of ammonium and the concentration of nitrate are monitored to be 3 mg/L or more, the anoxic stirring duration in the next cycle is prolonged until the concentration of total inorganic nitrogen is 5 mg/L or less; regulation of the C/N ratio and anoxic stirring duration enables the nitrate-dependent denitrifying bacteria to fully utilize organic matter to provide sufficient nitrite for the growth of anammox bacteria, and in turn, the anammox bacteria supply the nitrate required for the growth of nitrate-dependent denitrifying bacteria; when the effluent TIN concentration reduces to below 5 mg/L and the abundance of anammox bacteria is higher than 10⁷ copies/dry sludge by PCR, it is determined that the enhanced self-enrichment of anammox bacteria is achieved.

The method for enhancing self-enrichment of anammox bacteria by nitrate-dependent denitrifying bacteria symbiotic metabolism provided by the present disclosure has the following characteristics and advantages:

(1) The method utilizes partial denitrification that is easily-controlled, requires less carbon demand, and has lower sludge production compared to complete denitrification. It not only reduces the disposal cost of waste sludge, but also can stably provide nitrite required for anammox bacteria, thus providing ideal conditions for the self-enrichment of the anammox bacteria.

(2) Anammox bacteria are enriched in a partial denitrification system, which can provide stable substrate nitrite for anammox bacteria. On the other hand, the nitrate produced by anammox reaction can be reduced by denitrifying bacteria. In addition, partial denitrification consumes organic matter, eliminating the negative impact of organic carbon on anammox bacteria. Also, the symbiotic metabolism of denitrifying bacteria provides a favorable condition for growing anammox bacteria, which is greatly beneficial to the self-enrichment of anammox bacteria.

(3) By in situ self-enrichment of anammox bacteria in the partial denitrification system, the challenge in the anammox seeding sludge can be eliminated. Moreover, this could enhance the anammox activity and improve the nitrogen removal efficiency.

(4) Partial denitrification coupling with anammox does not require aeration and can reduce carbon source consumption. The self-enrichment of anammox bacteria can be enhanced by optimizing the key parameters, such as the C/N ratio and the anoxic stirring duration, making the control measures straightforward. In addition, there is no need to control temperature and pH, thus simplifying operations. Anammox bacteria can be effectively enriched under different influent concentrations, and achieve a good nitrogen removal efficiency, making the denitrification coupling with anammox widely applicable.

(5) Integrating the gas-liquid separation system and the hydraulic cyclone separation system in series after the enhanced self-enrichment system enables the separation of gas, liquid and solid in the effluent. This effectively retains the granular structure, prevents the loss of functional bacteria, and avoids the sludge float caused by the gas generated in the self-enrichment system. In addition, long-term monitoring of gas generation rate provides insights into the metabolism of the functional bacteria in the reaction, allowing for timely adjustments to the self-enrichment condition, thereby ensuring the efficient enrichment of anammox bacteria.

### BRIEF DESCRIPTION OF THE DRAWINGS

FIG. 1 is a device for enhancing self-enrichment of anammox bacteria by nitrate-dependent denitrifying bacteria symbiotic metabolism.

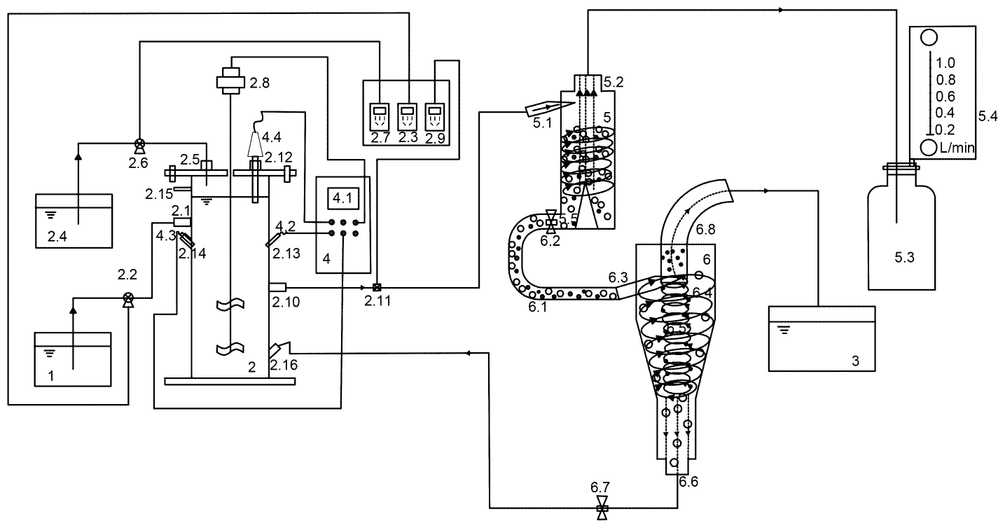

In FIG. 1: 1—influent tank; 2—enhanced anammox bacteria self-enrichment system; 3—effluent tank; 4—online real-time control system; 2.1—first wastewater inlet; 2.2—first peristaltic pump; 2.3—first time switch; 2.4—carbon source tank; 2.5—carbon source inlet; 2.6—second peristaltic pump; 2.7—second time switch; 2.8—stirrer; 2.9—third time switch; 2.10—effluent outlet; 2.11—effluent control valve; 2.12—sampling port; 2.13—first probe socket; 2.14—second probe socket; 2.15—overflow pipe; 2.16—recycled granules inlet; 4.1—display screen; 4.2—nitrate probe monitor; 4.3—ammonium probe monitor; 4.4—dissolved oxygen probe monitor; 5—gas-liquid separation system; 5.1—mixture inlet; 5.2—gas outlet; 5.3—gas collector; 5.4—gas flow detector; 5.5—discharge outlet; 6—hydraulic cyclone separation system; 6.1—U-shaped pipe; 6.2—first valve; 6.3—cyclone inlet; 6.4—outer cyclone; 6.5—inner cyclone; 6.6—granule discharge port; 6.7—second valve; 6.8—effluent outfall.

### DETAILED DESCRIPTION OF THE EMBODIMENTS

The present disclosure is further described with reference to the accompanying drawings and examples. As shown in FIG. 1, a device for enhancing self-enrichment of anammox bacteria by nitrate-dependent denitrifying bacteria symbiotic metabolism includes an influent tank (1), an enhanced anammox bacteria self-enrichment system (2), an effluent tank (3), an online real-time control system (4), a gas-liquid separation system (5), and a hydraulic cyclone separation system (6).
the enhanced anammox bacteria self-enrichment system (2) is provided with a first wastewater inlet (2.1), a first peristaltic pump (2.2), a first time switch (2.3), a carbon source tank (2.4), a carbon source inlet (2.5), a second peristaltic pump (2.6), a second time switch (2.7), a stirrer (2.8), a third time switch (2.9), an effluent outlet (2.10), an effluent control valve (2.11), a sampling port (2.12), a first probe socket (2.13), a second probe socket (2.14), an overflow pipe (2.15), and a recycled granules inlet (2.16). The online real-time control system is provided with a display screen (4.1), a nitrate probe monitor (4.2), an ammonium probe monitor (4.3), and a dissolved oxygen probe monitor (4.4). The gas-liquid separation system (5) is provided with a mixture inlet (5.1), a gas outlet (5.2), a gas collector (5.3), a gas flow detector (5.4), and a discharge outlet (5.5). The hydraulic cyclone separation system (6) is provided with a U-shaped pipe (6.1), a first valve (6.2), a cyclone inlet (6.3), an outer cyclone (6.4), an inner cyclone (6.5), a granule discharge port (6.6), a second valve (6.7), and an effluent outfall (6.8).

The influent tank (1) is connected to the first wastewater inlet (2.1) of the enhanced anammox bacteria self-enrichment system (2) through the first peristaltic pump (2.2), which is controlled by the first time switch (2.3). The carbon source tank (2.4) is connected to the carbon source inlet (2.5) of the enhanced anammox bacteria self-enrichment system (2) through the second peristaltic pump (2.6), which is controlled by the second time switch (2.7). The stirrer (2.8) is controlled by the online real-time control system (4) and connected by a wire. The nitrate probe monitor (4.2) is connected to the enhanced anammox bacteria self-enrichment system (2) through the first probe socket (2.13). The ammonium probe monitor (4.3) is connected to the enhanced anammox bacteria self-enrichment system (2) through the second probe socket (2.14). The dissolved oxygen probe monitor (4.4) is connected to the enhanced anammox bacteria self-enrichment system (2) through the sampling port (2.12), which is used to monitor the dissolved oxygen concentration that is controlled below 0.1 mg/L for the well anoxic environment. the enhanced anammox bacteria self-enrichment system (2) is connected to the gas-liquid separation system (5) through the effluent outlet (2.10) and the mixture inlet (5.1), and the process is generated through the effluent control valve (2.11), which is controlled by the third time switch (2.9). The gas-liquid separation system (5) is connected to the gas collector (5.3) through the gas outlet (5.2). The hydraulic cyclone separation system (6) is connected to the gas-liquid separation system (5) through the U-shaped pipe (6.1), which is controlled by a first valve (6.2). The hydraulic cyclone separation system (6) is connected to the enhanced anammox bacteria self-enrichment system (2) through the recycled granules inlet (2.16), which is controlled by a second valve (6.7). The hydraulic cyclone separation system (6) is connected to the effluent tank (3) through the effluent outfall (6.8).

The specific running process was as follows:

The method for enhancing self-enrichment of anammox bacteria by nitrate-dependent denitrifying bacteria symbiotic metabolism was as follows.

(1) Enrichment of nitrate-dependent denitrifying bacteria: excess sludge from municipal wastewater disposal treatment plants was inoculated into the enhanced anammox bacteria self-enrichment system, with the Suspended Solids (SS)) being 3,000-6,000 mg/L and the Volatile Suspended Solids (VSS) being 2,000-4,000 mg/L. The nitrate concentration in the influent tank ranges from 50 to 100 mg/L, and sodium acetate was added as an external carbon source. Each running cycle of the enhanced anammox bacteria self-enrichment system includes influent, carbon source addition, anoxic stirring, precipitation, drainage, and idle. After the influent and the carbon source addition, the C/N ratio in the enhanced anammox bacteria self-enrichment system was kept at 2.0-3.5. The drainage ratio was 40-70%. The anoxic stirring was performed at 80-140 r/min for 2-4 hours. The nitrate concentration was monitored through the nitrate probe monitor during running, and the data was uploaded to the online real-time control system. When the anoxic stirring duration reached 4 hours and the nitrate concentration was over 10 mg/L, the C/N ratio was increased in the next cycle. When the anoxic stirring duration reached 2 hours and the nitrate concentration was below 1 mg/L, the C/N ratio was reduced in the next cycle. When the transformation rate of nitrate into nitrite is 70% or more and the nitrate concentration in the effluent was 3 mg/L or less, the status was maintained stably for over 30 days. The microbial community of activated sludge was detected and analyzed by 16S rRNA high-throughput sequencing technology. When the relative abundance of one type of denitrifying bacteria reached over 30%, it was determined that the enrichment of nitrate-dependent denitrifying bacteria was successful.

(2) In situ self-enrichment of anammox bacteria: The influent of the enhanced anammox bacteria self-enrichment system was replaced with actual municipal wastewater and nitrate-containing wastewater, where the nitrate concentration in the nitrate-containing wastewater was 50-100 mg/L, and the ammonium concentration in municipal wastewater was 30-80 mg/L; the C/N ratio was adjusted to 2.0-2.8. The anoxic stirring duration in the enhanced anammox bacteria self-enrichment system was 6-10 hours. When the removal rate of nitrite and ammonium reached over 80% and 60%, respectively, the nitrate concentration in the effluent was below 5 mg/L, and the abundance of anammox bacteria was over 104 copies/dry sludge as detected by polymerase chain reaction (PCR), it was determined that the self-enrichment of anammox bacteria was successfully achieved. The granular sludge discharged from the effluent was recycled through the gas-liquid separation system and the hydraulic cyclone separation system connected to the enhanced anammox bacteria self-enrichment system, and was returned to the self-enrichment enhancement system to retain anammox bacteria. The effluent entered the gas-liquid separation system at first. According to different centrifugal forces on different phases, the gas was discharged upward into a collector equipped with a gas flowmeter for gas generation rate detection. The first valve was opened to make liquid phase enter the hydraulic cyclone separation system through a U-shaped pipe. Due to different inertia, flocs entered an inner cyclone separator with the light water phase and was discharged from an upper part into the effluent tank; the granular sludge entered the outer cyclone separator, and flowed out from the bottom. The second valve was opened to recycle and return the granular sludge into the enhanced anammox bacteria self-enrichment system.

(3) Enhanced self-enrichment of anammox bacteria by nitrate-dependent denitrifying bacteria symbiotic metabolism: Anammox bacteria were transitioned to a self-enrichment enhancement stage, the nitrate concentration in the influent was adjusted to 80-200 mg/L, the ammonium concentration was adjusted to 60-120 mg/L, the C/N ratio was adjusted to 2.0-2.5, and the anoxic stirring duration was 3-8 hours. When the ammonium concentration was 5 mg/L or more and the nitrate concentration was below 1 mg/L as monitored by the ammonium probe monitor and the nitrate probe monitor, the nitrate concentration in the influent was decreased in the next cycle until the concentration of total inorganic nitrogen was below 5 mg/L. When the ammonium concentration and the nitrate concentration were monitored to be 3 mg/L or more, the anoxic stirring duration in the next cycle prolonged until the concentration of the total inorganic nitrogen was below 5 mg/L. Based on the rational setting of the C/N ratio and the anoxic stirring duration, nitrate-dependent denitrifying bacteria can fully utilize organic matter to provide sufficient nitrite for the growth of anammox bacteria, and in turn, anammox bacteria supplied the nitrate required for the growth of nitrate-dependent denitrifying bacteria. When the total inorganic nitrogen concentration in the effluent was below 5 mg/L and the abundance of anammox bacteria was higher than 10⁷ copies/dry sludge by PCR, it is determined that the self-enrichment of anammox bacteria is enhanced.

(4) Results of continuous experiments have shown that nitrate-dependent denitrifying bacteria and anammox bacteria can successfully achieve self-enrichment, and the abundance of anammox bacteria can reach higher than 104 copies/dry sludge, and the self-enrichment is accelerated with the help of nitrate-dependent denitrifying bacteria symbiotic metabolism. When the system runs at a higher load and a lower C/N ratio, by continuously shortening the anaerobic stirring duration, the ammonium removal rate in the effluent can be more than 95%, and the nitrate removal rate can be more than 90%, thus achieving deep nitrogen removal without inoculating the anammox sludge.

What is claimed is:

1. A device for enhancing self-enrichment of anammox bacteria by nitrate-dependent denitrifying bacteria symbiotic metabolism, comprising an influent tank (1), an enhanced anammox bacteria self-enrichment system (2), an effluent tank (3), an online real-time control system (4), a gas-liquid separation system (5), and a hydraulic cyclone separation system (6); wherein
the enhanced anammox bacteria self-enrichment system (2) is provided with a first wastewater inlet (2.1), a first peristaltic pump (2.2), a first time switch (2.3), a carbon source tank (2.4), a carbon source inlet (2.5), a second peristaltic pump (2.6), a second time switch (2.7), a stirrer (2.8), a third time switch (2.9), an effluent outlet (2.10), an effluent control valve (2.11), a sampling port (2.12), a first probe socket (2.13), a second probe socket (2.14), an overflow pipe (2.15), and a recycled granules inlet (2.16); the online real-time control system is provided with a display screen (4.1), a nitrate probe monitor (4.2), an ammonium probe monitor (4.3), and a dissolved oxygen probe monitor (4.4); the gas-liquid separation system (5) is provided with a mixture inlet (5.1), a gas outlet (5.2), a gas collector (5.3), a gas flow detector (5.4), and a discharge outlet (5.5); the hydraulic cyclone separation system (6) is provided with a U-shaped pipe (6.1), a first valve (6.2), a cyclone inlet (6.3), an outer cyclone (6.4), an inner cyclone (6.5), a granule discharge port (6.6), a second valve (6.7), and an effluent outfall (6.8);
the influent tank (1) is connected to the first wastewater inlet (2.1) of the enhanced anammox bacteria self-enrichment system (2) through the first peristaltic pump (2.2), which is controlled by the first time switch (2.3); the carbon source tank (2.4) is connected to the carbon source inlet (2.5) of the enhanced anammox bacteria self-enrichment system (2) through the second peristaltic pump (2.6), which is controlled by the second time switch (2.7); the stirrer (2.8) is controlled by the online real-time control system (4) and is connected by a wire; the nitrate probe monitor (4.2) is connected to the enhanced anammox bacteria self-enrichment system (2) through the first probe socket (2.13); the ammonium probe monitor (4.3) is connected to the enhanced anammox bacteria self-enrichment system (2) through the second probe socket (2.14); the dissolved oxygen probe monitor (4.4) is connected to the enhanced anammox bacteria self-enrichment system (2) through the sampling port (2.12), which is used to monitor the dissolved oxygen concentration that is controlled below 0.1 mg/L for well anoxic conditions; the enhanced anammox bacteria self-enrichment system (2) is connected to the gas-liquid separation system (5) through the effluent outlet (2.10) and the mixture inlet (5.1), and the process is generated through the effluent control valve (2.11), which is controlled by the third time switch (2.9); the gas-liquid separation system (5) is connected to the gas collector (5.3) through the gas outlet (5.2); the hydraulic cyclone separation system (6) is connected to the gas-liquid separation system (5) through the U-shaped pipe (6.1), which is controlled by the first valve (6.2); the hydraulic cyclone separation system (6) is connected to the enhanced anammox bacteria self-enrichment system (2) through the recycled granules inlet (2.16), which is controlled by the second valve (6.7); the hydraulic cyclone separation system (6) is connected to the effluent tank (3) through the effluent outfall (6.8).

2. A method for enhancing self-enrichment of anammox bacteria by nitrate-dependent denitrifying bacteria symbiotic metabolism through using the device according to claim 1, comprising the following steps:
(1) enrichment of nitrate-dependent denitrifying bacteria: inoculating an excessive amount of sludge from municipal wastewater treatment plant into the enhanced anammox bacteria self-enrichment system, wherein the sludge has a concentration of 3000-6000 mg/L by suspended solids (SS), and a concentration of 2000-4000 mg/L by volatile suspended solids (VSS); adjusting a mass concentration of nitrate in the influent tank to 50-100 mg/L, adding sodium acetate as an external carbon source, and performing running cycles in the enhanced anammox bacteria self-enrichment system, wherein each cycle including influent, carbon source addition, anoxic stirring, precipitation, drainage and idle; wherein after the influent and the carbon source addition, a carbon-to-nitrogen (C/N) ratio in the enhanced anammox bacteria self-enrichment system is kept at 2.0-3.5; a drainage ratio is 40-70%; the anoxic stirring is performed at 80-140 r/min for 2-4 h; a nitrate concentration is monitored through a nitrate probe monitor during running, and data is uploaded to the online real-time control system; when the anoxic stirring reaches 4 h and the nitrate concentration is over 10 mg/L, the C/N ratio is increased in the next cycle; when the anoxic stirring lasts for 2 h and the nitrate concentration is below 1 mg/L, the C/N ratio is reduced in the next cycle; A condition wherein a transformation rate of nitrate into nitrite is 70% or more, and the nitrate concentration in the effluent is 3 mg/L or less is maintained stably for more than 30 days; a microbial community of activated sludge is detected and analyzed by 16SrRNA high-throughput sequencing technology; and when a relative abundance of one genus of denitrifying bacteria reaches over 30%, it is determined that the enrichment of nitrate-dependent denitrifying bacteria is successful;
(2) in situ self-enrichment of anammox bacteria: replacing influent of the enhanced anammox bacteria self-enrichment system with actual municipal wastewater and nitrate-containing wastewater, wherein the nitrate concentration in the nitrate-containing wastewater is 50-100 mg/L, and the ammonium concentration in the municipal wastewater is 30-80 mg/L; adjusting the C/N ratio to 2.0-2.8; extending the anoxic stirring duration in the enhanced anammox bacteria self-enrichment system to 6-10 h, wherein when a removal rate of nitrite and ammonium reaches over 80% and 60%, respectively, the nitrate concentration in the effluent is below 5 mg/L, and the abundance of anammox bacteria is more than 104 copies/dry sludge as detected by polymerase chain reaction (PCR), it is determined that the self-enrichment of anammox bacteria is achieved; recycling granular sludge discharged from the effluent through the gas-liquid separation system and the hydraulic cyclone separation system connected to the enhanced anammox bacteria self-enrichment system, and returning granular sludge discharged from the effluent to the self-enrichment enhancement system to retain the anammox bacteria, wherein effluent enters the gas-liquid separation system at first; according to different centrifugal forces on different phases, the gas is discharged upward into a collector equipped with a gas flowmeter for gas generation rate detection; the first valve is opened to make the liquid phase enter the hydraulic cyclone separation system through a U-shaped pipe; due to different inertia, floc sludge enters an inner cyclone separator with a light water phase and is discharged from an upper part into an effluent tank, and the granular sludge enters an outer cyclone separator, and flows out from the bottom; and the second valve is opened to recycle and return the granular sludge into the enhanced anammox bacteria self-enrichment system; and
(3) enhanced self-enrichment of anammox bacteria by nitrate-dependent denitrifying bacteria symbiotic metabolism: transiting the anammox bacteria to a self-enrichment enhancement stage, adjusting the nitrate concentration in the influent to 80-200 mg/L, adjusting the ammonium concentration to 60-120 mg/L, adjusting the C/N ratio to 2.0-2.5, and performing the anoxic stirring for 3-8 h, wherein, when the ammonium concentration is 5 mg/L or more and the nitrate concentration nitrate is 1 mg/L or less as monitored by an ammonium probe monitor and a nitrate probe monitor, the nitrate concentration in the influent is decreased in the next cycle until a concentration of total inorganic nitrogen is below 5 mg/L; when the ammonium concentration and the nitrate concentration are monitored to be both 3 mg/L or more, the anoxic stirring duration in the next cycle is prolonged until the concentration of the total inorganic nitrogen is below 5 mg/L; regulation of the C/N ratio and duration of anoxic stirring enables the nitrate-dependent denitrifying bacteria to fully utilize organic matter to provide sufficient nitrite for the growth of anammox bacteria, and in turn, the anammox bacteria supply the nitrate required for the growth of nitrate-dependent denitrifying bacteria; when the effluent TIN concentration is below 5 mg/L and the abundance of anammox bacteria is higher than 10⁷ copies/dry sludge by PCR, it is determined that the self-enrichment of anammox bacteria is enhanced.
